(19) United States
(12) Patent Application Publication
(10) Pub. No.: US 2011/0218616 A1
(43) Pub. Date: Sep. 8, 2011

(54) Cancer Stent Treatment Device Using Nanotechnology

(75) Inventor: Erwin Dale Thomas, Pomona, CA (US)

(73) Assignee: Erwin Dale Thomas, Pomona, CA (US)

(21) Appl. No.: 12/978,563

(22) Filed: Dec. 26, 2010

Related U.S. Application Data
(60) Provisional application No. 61/290,187, filed on Dec. 26, 2009.

(51) Int. Cl. A61F 2/82; B82Y 5/00

(52) U.S. Cl. 623/1.15; 977/742; 977/904

(57) ABSTRACT

This application is for a device using aptamers, multiwalled carbon nanotubes (MWCNTs) filled with nanosized iron oxide particles immobilized magnetically on a stent by a magnet and then subsequently killing circulating tumor cells (CTCs) attached to the aptamers by heating the carbon nanotubes with near infrared (NIR) lasers and then release from the stent by removing the permanent magnet and subsequent excretion of the MWCNTs.

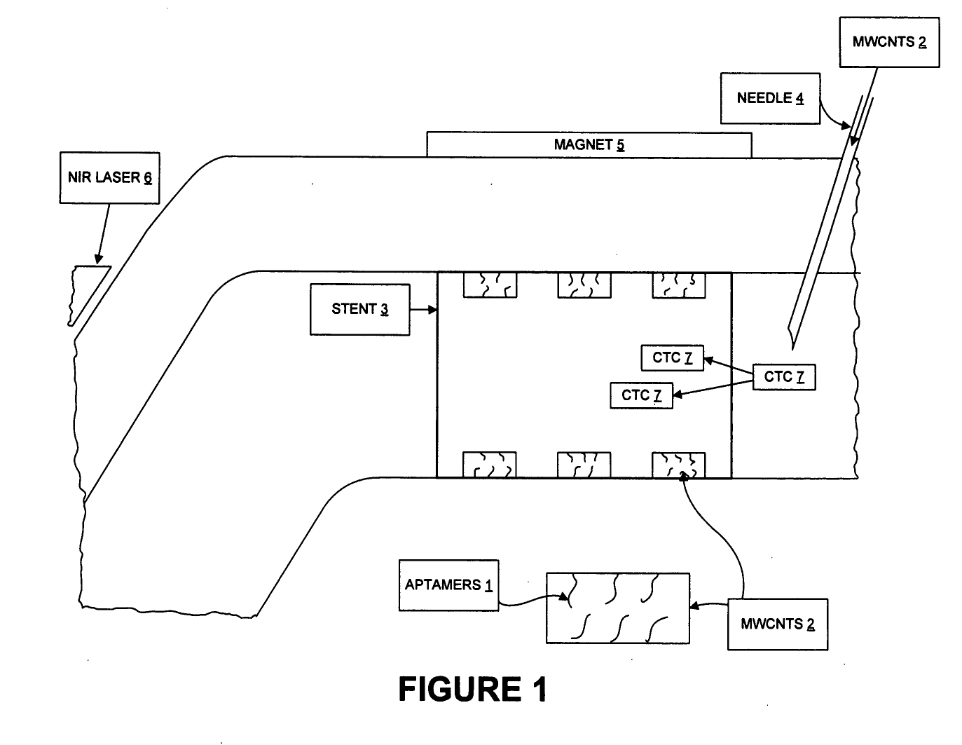

Provisional Patent 61/290,187 Filed 2009 Dec. 26 Stent Cancer Nanotechnology Platform

### BACKGROUND

Although up to 90% of deaths result from cancer result from metastasis and 90% of cancer treatment failures result from multi drug resistance, little if any literature describes a fully-functional in vivo nanotechnology treatment system. The problem is how to use nanotechnology to efficiently identify these cells specifically and comprehensively and then destroy them, especially over a long-period of time.

### SUMMARY

To address this problem, this application is for a device using aptamers, multiwalled carbon nanotubes (MWCNTs) filled with nanosized iron oxide particles immobilized magnetically on a stent by a magnet and then subsequently killing circulating tumor cells (CTCs) attached to the aptamers by heating the carbon nanotubes with near infrared (NIR) lasers and then release from the stent by removing the permanent magnet and subsequent excretion of the MWCNTs as is well-known.

### DETAILED DESCRIPTION

Targeting and capture. Cancer metastasizes, or cells (CTCs) break off from the parent cancer with the same tissue, they disperse through the blood and lymph to predictable, non-random locations due to both adhesive interaction of selectins, chemokines, and integrins, between the cancer and blood vessels in target tissue and mechanical forces. The circulating tumor cells are of low frequency, between 1 and several hundred per milliliter of blood or about 5,000 to 1,000,000 total in 5 liters,ⁱ and can be identified and bound to delivery vehicles to over expressed antigen membrane markers, or ligands, including Ep-CAM, HER-2, folic acid receptor, VIP-R, or whole cell identificationⁱⁱ using antibodies, proteins, aptamers, or other molecules.^{iii, iv} In addition to active targeting CTC ligands, to attract the CTCs significantly more strongly than natural metastasis target locations^{v}, increasing the frequency and duration of CTC contacts by having a fairly high-flow location and increased surface area or concentration of contacts can increase target cell capture by 1000%.^{vi}

Durability: Targeted delivery devices have been shown to not need long contact times to detect and attach to tumors^{vii}. The proposal is for an implanted device to attach to and immobilize passing CTCs. For the device to survive the longest, the strongest and fewest bonds should be used to hold it together. Additionally, making the particles as small as possible, functionalizing them with aptamers, and possibly giving them a positive charge, decreases the possibility of reticuloendothelial system (RES) attack and increase the clearance rate and likelihood.^{iv, viii} Thus, instead of outside attachment, if magnetic iron oxide nanoparticles are inside CNTs, this allows all of the bonding to be for attachments to the target CTCs.

Cargo. By design, the MWCNTs are attached to the outside of the CTCs avoiding (1) multidrug resistance reaction of the CTCs where CTCs attempt to eject substances entering their structure,^{ix} (2) toxicity of possible toxic cargo entry into non-targeted cells and (3) any problems associated with internalization of cargo by the cells (e.g. siRNA having unintended consequences if too many enter the cell).

Implantation and operation. To kill cells, one can heat the MWCNTs using near infrared (NIR) lasers in the non-body absorbing wavelength of 808 nm to a high enough temperature to kill attached cells.^{x}

STENT Coating of CNTs: Magnetic particles have been shown to evenly coat the inside of a stent with cells in vivo.^{xi} Instead of cells, the iron oxide in this case would be encapsulated in the MWCNTs to allow injection and immobilized on the stent for both replenishment and clearance. A stent as the immobilizing platform is well tested and widely used for other purposes.

### Specification

This is but one example of an embodiment of this invention. The preferred embodiment could be as follows, but not limited to:

Structure: The nanostructures are approximately 150 nm diameter, 200 nm long MWCNTs (2) filled with magnetic iron oxide nanoparticles made as previously described.^{xii} These are immobilized on a stainless steel stent (3), with nanopillars made as previously described,^{xiii} using as previously described for cells with iron oxide particles.^{viii}

Functionalization: The MWCNTs are functionalized by charging them and having thiolated aptamers attached with possibly multiple types of bonds including covalently and pi-bond stacking as previously described.^{ii, xiv, xv, xvi}

Capturing Mechanism: The capturing mechanism is aptamers, developed in vivo as previously described,^{xix} for whole cancer cells, and may also target the membrane bound ligand folate as this is not expressed in other cells in the blood.^{vii}

Implant: Multiple stainless steel stents may be 10 mm long are implanted in a large, accessible vein like the wrist (e.g. median ante brachial vein or cephalic vein) where a watch like device to hold a permanent magnet of maybe 1,000 gauss could be worn to magnetize the stent in a manner previously described. The MWCNTs could be injected just upstream from the stents with a magnet over the stent to attract and immobilize the particles as previously described done evenly and completely as previously described for distributing cells onto a stent in vivo.^{vii} NIR imaging (not pictured) could be done to ensure effective implantation of the MWCNTs as previously described for SWCNTs.^{xvii}

Destruction Mechanism: Instead of internalization into the CTCs, the MWCNTs would be attached to the outside of the CTCs. To kill the CTCs, the MWCNTs could be targeted by an NIR laser of approximately 850 nm and heated enough to kill attached cancer cells even though the MWCNTs are not internalized by the cell (they used SWCNTs).^{x} Measurement of effectiveness of treatment could be measured by periodic drawing of blood and testing with cytometry as described.^{xviii}

Clearance and Replenishment Mechanism: To clear the MWCNTs with attached killed CTCs, the permanent magnet could be gradually removed from over the stents, and the MWCNTs allowed to circulate until clearance by the liver and spleen.^{xix} The magnets over each stent could then be re-applied on the skin and MWCNTs injected again.

### Analysis:

There is some suggestion in the literature that aptamers, especially developed in vivo^{xx}, might be more stable with temperature/pH/salts, be an order of magnitude smaller,^{xxi}, may be more specific and attractive than antibodies for CTCs that need to distinguish from serum antigens and other tissue encountered in the blood or lymph, may be tailored specifically from individual patient tumors, may have less false positives and negatives than antibodies, and easier to manipulate and modify.^{xxii,xxiii,xxiv, xxv, ii, iv, xvi, xvii} Antibodies for instance targeting folate receptors, could however also be used. Adding competing selectin/chemokine^{xxvi} attractants to the MWCNTs is not likely helpful as these molecules are part of significant normal body chemistry that one would not want to alter. To further increase the attractiveness the surface area and thus the frequency and duration of CTC contact, MWCNTs are used being about 150 nm thick and additionally the stents are lined with nanopillars.^{vi} PEGalation is not used to save bonds for aptamers and these are intended to reduce as much as possible attack by the RES. It is thought to use as many of the bonds as possible for binding and immobilizing the CTCs as the more bonds to the MWCNTs, the more unstable the pi bonds may become. Direct attachment of aptamers to the MWCNTs was chosen as possibly more stable than dendrimer attachment. MWCNTs probably are better delivery vehicles because (1) carbon nanotubes can be heated effectively for cancer killing, they can be imaged, they can be filled with magnetic iron oxide, their large size may reduce cellular uptake by unintended targets, and aptamers can be attached to them. The downside is that MWCNTs are large so more difficult to excrete. Membrane ligand or cell targeting was chosen over pH or enzyme targeting^{xxvii,iv} as in the blood or lymph, pH or enzyme changes may be less noticeable. Magnetic ferrous oxide particles were chosen as they are non-toxic, small, cheap, and easy to use.^{vii} NIR ablation was selected as it is simple, proven, can be controlled, and evades any problems of cell entry and endosomal release, multi-drug resistance, cell unanticipated functionality targeting, and unanticipated toxicity to non-targeted cells which might be an issue of si-RNA/drugs used. Clearance and replenishment by magnetic particles and magnets was chosen to significantly ease the process instead of surgery. NIR imaging of MWCNTs was chosen instead of quantum dots to conserve bonding sites for aptamers.

### Addressing Pitfalls:

Potential problems include non-specific target binding. This could be addressed by using multiple aptamers that target different ligands, less specific binding by possibly attaching PEG groups to the aptamers, using antibodies in addition to aptamers, or trying to incorporate pH activated targeting.^{iv} If the MWCNT structure does not bind the CTCs strongly enough, the device won't work. Increasing magnet strength, changing the structure to maybe dendrimers with attached gold particles and/or iron oxide particles for NIR heating and magnetic binding and imaging could be tried. Different sized MWCNTs could be tried to increase the binding effectiveness. The magnet could interfere with body function or not be effective immobilizer which could be addressed by changing the strength. Clearance may not work as the MWCNTs may be too large. Smaller or SWCNTs might be tried instead. Gold covered CNTs might be tried though they might not respond to NIR imaging. Gold might reduce aggregation which might improve clearance. Clearance might be improved by slowly removing the magnet so that the particles with attached cancer cells are slowly released over several days. The urine probably should be collected separately as Nan particles in the environment may be dangerous. Quantum dots might be attached to the MWCNTs if MWCNT NIR imaging is ineffective.

### Testing:

This could be tested first on mice over several months to determine effectiveness of magnetic anchoring of MWCNTs, specific targeting, durability of the structure, effectiveness of killing and clearance, long-term changes on red blood cells of long-term magnetism, RES reactions, and accumulation of MWCNTs in the liver or spleen. Imaging by NIR could determine MWCNT coverage of the stent as previously described.^{vii} Blood draws to determine concentration of CTCs and effectiveness of removal. Clearance effectiveness could be tested by checking collecting urine samples after removal of the magnets. These issues could be subsequently tested in humans in different blood vessels.

### Abstract:

This application is for a device using aptamers, multiwalled carbon nanotubes (MWCNTs) filled with nanosized iron oxide particles immobilized magnetically on a stent by a magnet and then subsequently killing circulating tumor cells (CTCs) attached to the aptamers by heating the carbon nanotubes with near infrared (NIR) lasers and then release from the stent by removing the permanent magnet and subsequent excretion of the MWCNTs.
ⁱ Nagrath, S.; Sequist, L. V.; Maheswaran, S.; Bell, D. W.; Irimia, D.; Ulkus, L.; Smith, M. R.; Kwak, E. L.; Digumarthy, S.; Muzikansky, A.; Ryan, P.; Balis, U. J.; Tompkins, R. G.; Haber, D. A.; Toner, M.; Isolation of rare circulating tumour cells in cancer patients by microchip technology, Nature, 2007, 450, 1235-1241.
ⁱⁱ Liu, G.; Mao, X.; Phillips, J. A.; Xu, H.; Tan, W.; Zeng, L; Aptamer-Nan particle strip biosensor for sensitive detection of cancer cells, Anal. Chem, 2009, pre-publication.
ⁱⁱⁱ Peer, D.; Karp, J. M.; Hong, S.; Farokhzad, O. C.; Margalit, R.; Langer, R.; Nanocarriers as an emerging platform for cancer therapy, Nature Nanotechnology, 2007, 2, 751-760.
^{iv} Gullotti, E.; Yeo, Y.; Extracellularly Activated Nan carriers: A New Paradigm of Tumor Targeted Drug Delivery, Molecular Pharmaceutics, 6(4) 1041-1051.
^{v} El-Sayed, I. H.; Huang, X.; El-Sayed, M. A.; Surface Plasmon Resonance Scattering and Absorption of anti-EGFR Antibody Conjugated Gold Nanoparticles in Cancer Diagnostics; Applications in Oral Cancer, Nano Letters, 2005, 5, 829-834.
^{vi} Wang, S.; Wang, H.; Jiao, J.; Chen, K.; Owens, G. E.; Kamei, K.; Sun, J.; Sherman, D. J.; Behrenbruch, C. P.; Wu, H.; Tseng, H., Three-dimensional nanostructured substrates toward efficient capture of Circulating Tumor Cells, Anew. Chem, 2009, 121, 9132-9135.
^{vii} Glanzha, E. Il; Shashkov, E. V.; Kelly, T.; Kim, J; Yang, L.; Zharov, V. P. In Vivo magnetic enrichment and multiplex photo acoustic detection of circulating tumour cells. Nature Nanotechnology. 2009, 333, 1-6.
^{viii} Kostarelos, K.; Bianco, A., Prato, M.; Promises, facts and challenges for carbon nanotubes in imaging and therapeutics, Nature Nanotechnology, 2009, 4, 627-633.
^{ix} Chen, B.; Lai, B.; Cheng, J.; Xia, G.; Gao, F.; Xu, W.; Ding, J.; Gao, C.; Sun, X.; Xu, C.; Chen, W.; Chen, N.; Liu,L; Li, X.; Wang, X.; Daunarubicin-loaded magnetic Nan particles of “Fe3O4 overcome multidrug resistance and induce apoptosis of K562-n/VCR cells in vivo, In. J. Nanomedicine, 2009, 4, 201-208.
^{x} Xiao, Y.; Gao, X.; Taratula, O; Treado, S.; Urbas, A.; Holbrook, R. D.; Cavicchi, R. E.; Avedisian, C. T.; Mitra, S.; Savla, R.; Wagner, P. D., Srivastava, S.; He, H.; Anti-HER@ IgY antibody-functionalized single-walled carbon nanotubes for detection and selective destruction of breast cancer cells, BMC Cancer, 2009, 9, 351-362.
^{xi} Polyak, B.; Fishbein, Il; Chorny, M.; Alferiev, I.; Williams, D.; Yellen, B.; Friedman, G.; Levy, R. J.; High field gradient targeting of magnetic Nan particle-loaded endothelial cells to the surfaces of steel stents, Proceedings of the National Academy of Sciences, 2008, 105-698-703.
^{xii} Narayanan, T. N.; Mary, A. P; Shaijumon, M. M.; Ci, L.; Ajayan, P.M.; Anantharaman, M. R.; On the synthesis and magnetic properties of multiwall carbon nanotube-super paramagnetic iron oxide Nan particle Nanocomposites, Nanotechnology, 2009, 20, 55607.
^{xiii} Loya, M. C.; Park, E.; Chen, L. H.; Bramman, K. S.; Jin, S.; Radially arrayed nanopillar formation on metallic stent wire surface via radio-frequency plasma, Acta Biomarterialia, 2009, in press.
^{xiv} Herrero, M. A.; Toma, F. M.; Al-Jamal, K. T.; Kostarelos, K.; Bianco, A.; Ros, T. D.; Bano, F.; Casalis, L.; Scoles, G.; Prato, M., Synthesis and Characterization of a Carbon Nanotube-Dendron Series for Efficient siRNA Delivery, J. Am. Chem. Soc., 2009, 131, 9843-9848. ^{xv} Zelada-guilllen, G. A.; Riu, J.; Duzgun, A.; Rius, F. X.; Immediate Detection of Living Bacteria at Ultralow concentrations using a carbon nanotube based potentiometric aptasensor, Angewandte Chemie, 2009, 48, 7334-7337.
^{xvi} Johnson, R.R.; Johnson, C.; Klein, M. L.; Probing the Structure of DNA-Carbon Nanotube Hybrids with Molecular Dynamics, Nano Letters, 2008, 8, 69-75.
^{xvii} Welsher, K. Liu, Z.; Sherlock, S. P.; Robinson, J. T.; Chen, Z.; Daranciang, D.; Dai, H.; A route to brightly flourescent carbon nanotubes for near-infrared imaging in mice, Nature Nanotechnology, 2009, 294, 773-780.
^{xviii} He, W.; Wang, H.; Hartmann, L. C.; Cheng, J.; Low, P. S.; In vivo quantization of rare circulating tumor cells by multiphase interracial flow cytometry, Proceedings National Academy of Sciences, Jul. 10, 2007, 104, 11760-11765.
^{xix} Kim, S; Garg, H.; Joshi, A.; Manjunath, Strategies for targeted nonverbal delivery of siRNAs in vivo, Cell, 2009, 491-500.
^{xx} Mi, J; Liu, Y.; RabbanI, Z. N.; Yang, Z.; Urban, J. H.; Sullenger, B. A.; Clary, B. M.; In vivo selection of tumor-targeting RNA motifs, nature chemical biology, 2009, 277, 1-3).
^{xxi} Zhou, J.; Soontornworajit, B.: Martin, J.; Sullenger, B. A.; Gilboa, E.; Wang, Y. A Hybrid DNA Aptamer-Dendrimer Nanomaterial for Targeted Cell Labeling, Macromolecular Bioscience, 2009, 9, 831-835.
^{xxii} Gomaa, A. I.; Khan, S. A.; Leen, E. L S.; Waked, I.; Taylor-Robinson, S.D.; Diagnosis of hepatocellular carcinoma, World J Gastroenterol, 2009, 15(11), 1301-1314.
^{xxiii} Xu, Y; Phillips, J. A.; Yan, J.; Li, Q.; Fan, Z. H.; Tan, W.; Aptamer-based Microfluidic Device for Enrishment, Sorting, and Detection of Multiple Cancer Cells, Analytic Chemistry, 2009, 81, 7436-7442.
^{xxiv} Shangguan, D.; Cao, Z.; Meng, L; Mallikaratchy, P.; Sefah, K.; Wang, H.; Li, Y.; Tan, W.; Cell-specific Aptamer Probes for Membrane Protein Elucidation in Cancer Cells, J of Proteome Research, 2008, 7, 2133-2139.
^{xxv} Berezovski, M. V., Lechmann, M.; Musheev, M. U.; Mak, T. W.; Krylov, S. N.; Aptamer-facilitated Biomarker Discovery (AptaBiD), J. American Chemical Society, 2008, 130, 9137-9143.
^{xxvi} Gout, S.; tremblay, P.; Selectins and selectin ligans in extravasation of cancer cells and organ selectivity of metastasis, Clin Exp Metastasis, 2008, 25, 335-344.
^{xxvii} Lee, E. S.; Gao, Z, Kim, D.; Park, K.; Kwon, I. C.; Bae, Y. H.; Super pH-sensitive Multifunctional Polymeric Micelle for Tumor pH Specific TAT Exposure and Multidrug Resistance, J. Control Release, 2008, 129, 228-236.

1. Nanotechnology devices or particles including but not limited to dendrimers, carbon nanotubes, or quantum Dots attached to a stent.

2. (canceled)

3. (canceled)

4. (canceled)

5. (canceled)

6. (canceled)

7. A device or procedure to kill cancer cells, that have been attached to a stent, by heating it.

8. (canceled)

9. A Cancer treatment device using increased inside stent surface area.

10. (canceled)
